(19) United States
(12) Patent Application Publication
(10) Pub. No.: US 2006/0293608 A1
(43) Pub. Date: Dec. 28, 2006

(54) Device for and method of predicting a user's sleep state

(75) Inventors: Daniel Rothman, Hopewell Jct., NY (US); Benjamin S. Rubin, Reading, MA (US); Paolo DePetrillo, Cranston, RI (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Axon Sleep Research Laboratories, Inc., Providence, RI (US)

(21) Appl. No.: 11/069,934

(22) Filed: Feb. 28, 2005

Related U.S. Application Data
(60) Provisional application No. 60/548,228, filed on Feb. 27, 2004.

(51) Int. Cl. A61B 5/04; G04F 8/00; G04F 10/00; G04B 47/00

(52) U.S. Cl. 600/545; 600/546; 368/9; 368/10

(57) ABSTRACT

A device and a method for waking a user in a desired sleep state. The device may predict an occurrence when the user will be in the desired sleep state, such as light sleep, and wake the user during that predicted occurrence. In one embodiment, a user may set a wake-up time representing the latest possible time that the user would like to be awakened. The occurrence closest to the wake-up time when the user will be in light sleep may be predicted, thereby allowing the user to sleep as long as possible, while awakening in light sleep. To predict when the user will be in the desired sleep state, the user's sleep state may be monitored during the night or sleep experience and the monitored information may be used in predicting when the user will be in the desired sleep state.

### RELATED APPLICATIONS

This application claims the benefit under 35 U.S.C. § 119(e) of U.S. Provisional Application Ser. No. 60/548,228, entitled “Sleep Phase Monitor and User Responsive Awakening Device and Methods for Using Same,” filed on Feb. 27, 2004, which is incorporated herein by reference in its entirety.

### FIELD OF THE INVENTION

The invention relates to predicting sleep states, more particularly to an alarm device for and a method of predicting a sleep state of a user and waking the user.

### BACKGROUND OF THE INVENTION

Sleep is the body's way of rejuvenating itself and is critical for learning and memory of both physical and mentally demanding tasks. Sleep is not an all-or-none phenomenon, however. Using electroencephalogram (EEG) analysis, sleep researchers have identified several distinct phases of sleep ranging from light dozing to deep sleep. These phases are marked by differing brain wave speeds and reflect different mental processes that are occurring while we sleep. For example, we dream during rapid eye movement (REM) sleep, which is characterized by rapid eye movements.

Currently, people are sleeping far less than the suggested optimal amount of an average of 8.4 hours per night due to a variety of factors, such as increased work hours, second or third jobs, longer commutes, increased media options, such as satellite television or internet websites, and family commitments. If people were getting enough sleep in their daily lives there would be little use for alarm clocks, as we would awaken naturally once the body had received enough sleep. However, since people are cutting into their optimal levels of sleep, alarm clocks are necessary to prematurely awaken sleepers.

Sleep Inertia is a phenomenon resulting from waking up without having had sufficient sleep and is roughly caused by the persistence of the physical stages of sleep into a waking state. For curtailed sleep, one of the most critical factors in determining the duration of Sleep Inertia is the sleep stage immediately preceding awakening. Abrupt awakening during deep sleep (e.g., Slow Wave Sleep or stage 3 or 4 sleep) produces greater Sleep Inertia than awakening during REM sleep or during light sleep (e.g., stage 1 or 2 sleep).

In addition, sleep debt caused by prior sleep deprivation prolongs the effects of Sleep Inertia. There is no direct evidence that Sleep Inertia is affected by a circadian rhythm. Circadian rhythm in this context means the natural twenty-four hour cycle that the human body exhibits with or without the presence of external stimuli, such as light. It seems that Sleep Inertia is more intense when awakening occurs near the core body temperature circadian trough than near its circadian peak.

A more controversial issue concerns the time course of Sleep Inertia. In a fully rested person awakened during the wrong stage of sleep, duration of Sleep Inertia may rarely exceed 30 minutes. However, because the average working person is carrying a large sleep debt, realistically, the duration of Sleep Inertia may exceed 3.5 hours, depending on the sleep state immediately preceding awakening. A more conservative middle ground estimates the duration of Sleep Inertia to be between one and two hours.

Previous attempts to remediate this problem include the “Zen alarm clock” and several “artificial dawn” clocks. The principle underlying both of these devices is first to elevate the sleeper to light sleep and then to awaken the sleeper. The Zen alarm clock uses a gradually series of bells, while the artificial dawn clock's stimulus is light of gradually increasing intensity.

Other wake-up devices include alarm clocks that detect the user's sleep state and awaken the user when the user is in light sleep, not during deep or REM sleep. One of the downsides to this method is that there is no absolute wake-up time, so this device is useless to someone who has to wake up by a certain time.

### SUMMARY OF THE INVENTION

At least one aspect of the invention provides a device and method of waking a user that may predict when a user may be in a desired sleep state. The device and method may allow the user to sleep as close to a wake-up condition as possible, while awakening the user during the desired sleep state. The device and method may also include a fail-safe, such that if a user does not enter the desired sleep state before the wake-up condition elapses, a user may be woken at the end of the wake-up condition.

In one illustrative embodiment, a method of waking a user is provided. The method includes determining a wake-up condition relating to when a user will be awakened, predicting at least one occurrence when the user will be in a desired sleep state, and waking the user. The predicted at least one occurrence is approximately at, near to, or during the wake-up condition.

In another illustrative embodiment, a wake-up device is provided. The wake-up device includes a monitoring portion producing an output and an alarm portion to receive the output. The monitoring portion may monitor at least one biosignal of a user during at least a portion of a sleep cycle of a user and predict a time period during which the user will be in a desired sleep state. The alarm portion may receive the output and assist in awakening the user.

In yet another illustrative embodiment, a use of material, including a metal, for a sensor for monitoring a sleep state of the user is provided.

Various embodiments of the present invention provide certain advantages. Not all embodiments of the invention share the same advantages and those that do may not share them under all circumstances.

Further aspects of the present invention, as well as the structure of various embodiments of the present invention are described in detail below with reference to the accompanying drawings.

### BRIEF DESCRIPTION OF DRAWINGS

The accompanying drawings are not intended to be drawn to scale. In the drawings, similar features are represented by like reference numerals. For clarity, not every component is labeled in every drawing. In the drawings:

FIG. 1 is a perspective view of an illustrative embodiment of a wake-up device being used by a user;

FIG. 2 is a perspective view of an illustrative embodiment of a headband unit of a wake-up device being worn by a user;

FIG. 3 is an exploded view of an illustrative embodiment of a headband unit of a wake-up device;

FIG. 4 is a schematic representation of an illustrative embodiment of a hypnogram;

FIG. 5 is a schematic block diagram of an illustrative embodiment of a wake-up device;

FIG. 6 is a schematic block diagram of an illustrative embodiment of a sensor portion of a headband unit;

FIG. 7 is a schematic block diagram of an illustrative embodiment of an alarm clock unit;

FIG. 8 is a schematic representation of an illustrative embodiment of a wake-up algorithm;

FIG. 9 is a schematic representation of an illustrative embodiment of a sleep state detection algorithm; and

FIG. 10 is a schematic representation of an illustrative embodiment of a hypnogram and delta activity, slow wave sleep (SWS) and spindle and/or k-complex activity during sleep.

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention are described below with reference to illustrative embodiments. It should be understood that reference to these illustrative embodiments is not made to limit aspects of the invention in any way. Instead, illustrative embodiments are used to aid in the description and understanding of various aspects of the invention. Therefore, the following description is intended to be illustrative, not limiting.

In one aspect of the invention, a device can predict an occurrence when a user will be in a desired sleep state, such as light sleep, and awaken the user during the predicted occurrence. A wake-up condition may also be established that defines one or more criteria regarding when the user should be awakened. For example, a user may indicate a wake-up condition as a set time, e.g., 7:00am, and an occurrence when the user is in light sleep (the desired sleep state) nearest to, or at, 7:00am may be predicted. The user may enter light sleep more than once during a sleep period (e.g., over the course of a night), and so that occurrence closest to the wake-up condition (e.g., 7:00am) when the user is in light sleep may be selected as a time when the user should be awakened.

In one embodiment, the user may set a wake-up time (a wake-up condition) representing the latest possible time that the user would like to be awakened. The occurrence of a desired sleep state that is closest to, but not after, the wake-up time may be predicted, thereby allowing the user to sleep as long as possible, while awakening in the desired sleep state. Since a user usually spends more than a moment in each sleep state, the predicted occurrence may itself be a window of time. The user may be awakened at the beginning of, during or at the end of the predicted occurrence. To predict when the user will be in light sleep, the user's sleep state may be monitored during sleep and the monitored information may be used in predicting when the user will be in the desired sleep state. In addition or alternatively, a sleep history of a user and/or a pre-programmed algorithm may be utilized in predicting when the user will be in light sleep or some other desired sleep state.

In another aspect of the invention, the user's sleep state may be monitored during the predicted occurrence, to ensure that the user is, in fact, in light sleep or another desired sleep state before the user is awakened. If the user is detected to be in the desired sleep state at the predicted occurrence, the user may be awakened. If the user is not detected to be in the desired sleep state at the predicted occurrence, the user's sleep state may continue to be monitored and if the user enters the desired sleep state, the user may be awakened. If the user does not enter the desired sleep state before the wake-up time, the user may be awakened at the wake-up time. In some embodiments, the sleep state of the user may be actively monitored throughout sleep, such that occurrences when the user may be in the desired sleep state may be continually predicted. The user's sleep state may be actively monitored for a portion or portions of sleep or for the entire sleep, up to and including a wake-up time, if used.

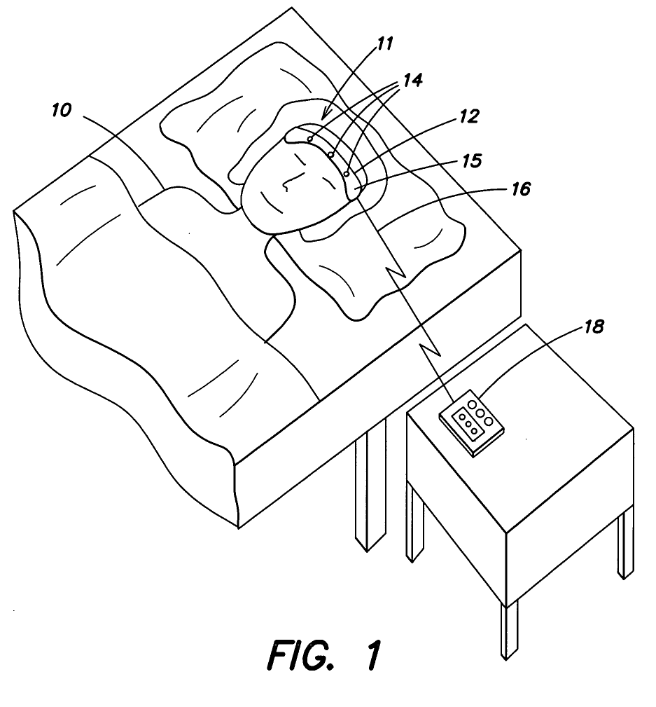

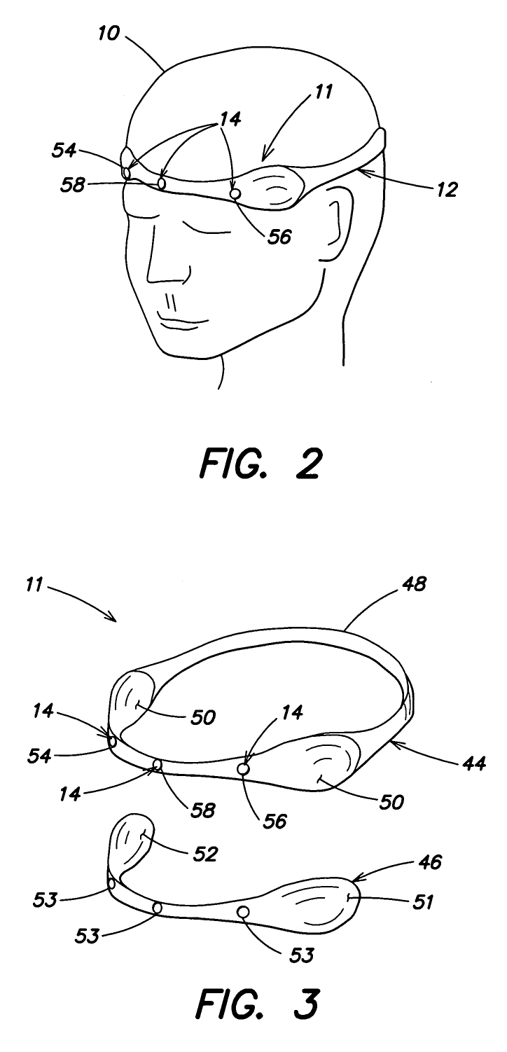

FIGS. 1 and 2 show an illustrative embodiment of a wake-up device in accordance with the invention. In this embodiment, the wake-up device may include a monitoring portion 11 and an alarm portion 18. Monitoring portion 11 may include one or more sensors, such as electrodes 14, for monitoring a biosignal of the user 10, and a device 15 including electronic circuitry and/or other components to predict an occurrence when the user 10 may be in a desired sleep state. Although the monitoring portion 11 need not necessarily contact the user 10, in this embodiment the monitoring portion includes a headband 12 that helps to position the electrodes 14 near targeted portions of the user's head. Other sensor arrangements may be used, whether contacting or non-contacting, to detect one or more biosignals of the user, such as body temperature, temperature gradients, blood pressure, galvanic skin response, eye or other body movement, etc. Two of the electrodes 54, 56 in this embodiment may be signal electrodes placed above the left and right eyes of the user, and a third electrode 58 may be a ground or reference electrode placed in the center of the forehead, as shown in the embodiment depicted in FIG. 2. However, any number of sensors may be used in any location as the present invention is not intended to be limited in this respect.

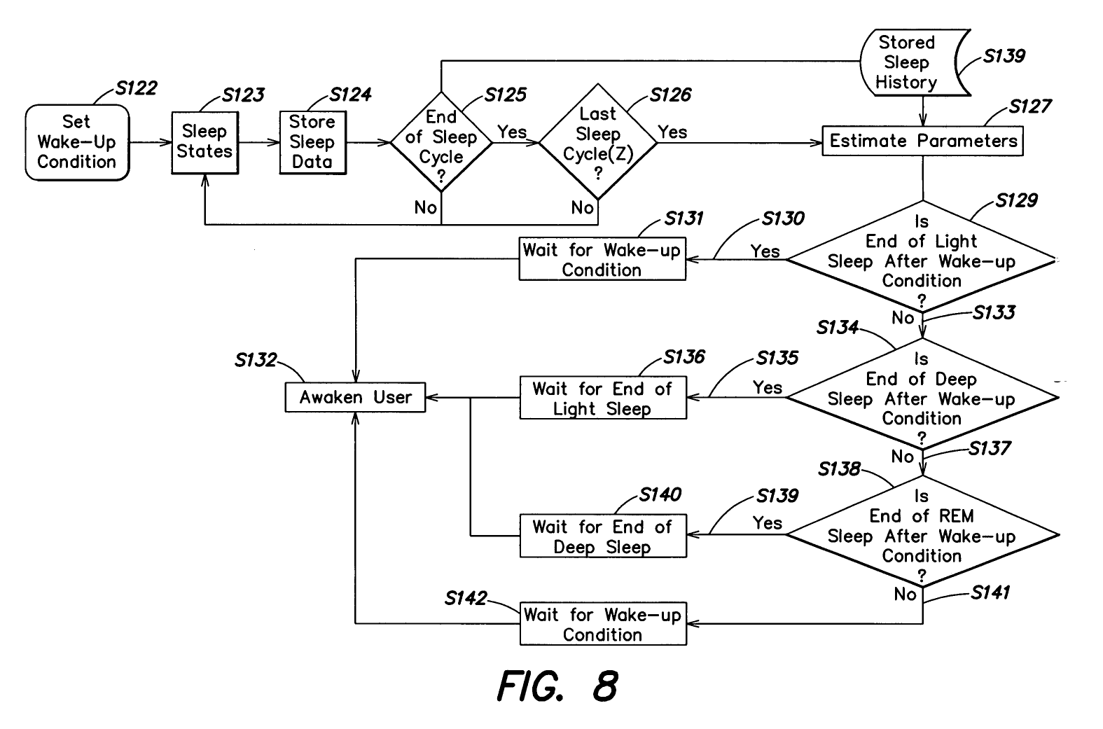

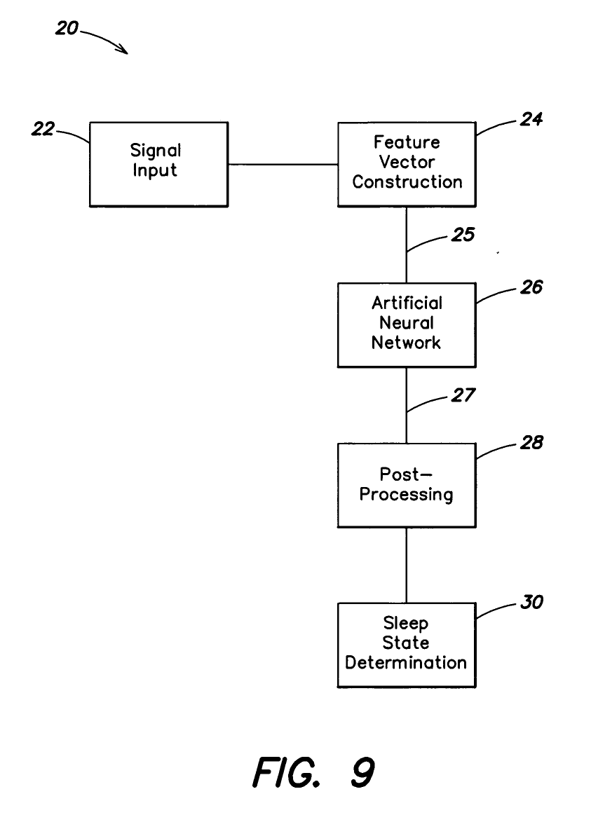

The device 15 may process information from the sensors 14 using any suitable algorithm, for example, the sleep state detection algorithm shown in FIG. 9, to determine the sleep state of the user. In addition, the device 15 may predict when the user will be in a desired sleep state, for example using the wake-up algorithm shown in FIG. 8. Based on the predicted occurrence of the user's desired sleep state, the device 15 may send an output to the alarm portion 18 indicating, for example, a time in the future when to sound an alarm to wake the user 10. The monitoring portion 11 and the alarm portion 18 may communicate in any suitable way, such as by wired or wireless link 16 (which may include any suitable communication network(s)). Based on information from the monitoring portion 11, the alarm portion 18 may awaken the user 10 using any means, such as a buzzer, a radio, a flashing light, and/or any other suitable means. It should be understood that the alarm portion 18 may be located in any suitable location, such as integrated with the monitoring portion 11 on the headband 12, in contact with one or more portions of the user 10, or located remotely from the user 10 and the monitoring portion 18. In addition, the device 15 may be located remotely from the sensor portion of the monitoring portion 11, such as in the alarm portion 18, at an Internet web site, at a local computer, etc.

In one embodiment, as shown in FIG. 3, the monitoring portion 11 may include an outer sleeve 44 and an inner sleeve 46. Outer sleeve 44 may include a headband strap 48, which may be adjustable to accommodate a variety of head sizes and shapes, for example, by having an elastic portion. Outer sleeve 44 may be molded from a resin or silicone-like material and may act to protect the device 15 and/or electrodes 14, for example, from moisture or from impacts, such as from being bumped or dropped. Outer sleeve 44 may contain one or more bulges 50, which may accommodate one or more portions of the device 15. Outer sleeve 44 may be attached to inner sleeve 46 by any means, such as by pins, hook and loop fasteners, buttons, snaps, fasteners, or any other means of permanent or detachable attachment.

Inner sleeve 46 may be made from a comfortable, breathable mesh and may include a disposable layer or pad. Inner sleeve 46 may provide padding for comfort or increased sanitation by being removable and/or washable. As shown in the embodiment in FIG. 3, electrodes 14 may be located on an outer side 51 of inner sleeve 46 but may extend through holes 53 to an inner side 52 of inner sleeve 46; through these holes 53, electrodes 14 may make contact with user 10. The sensors may be located on any side of any sleeve or be positioned therebetween, as the present invention is not intended to be limited in this respect.

It should be appreciated that one or more sleeves may be completely separable from the device 15 and/or the electrodes 14, such that the sleeves may be washed. In addition or alternatively, the one or more sleeves may be disposable, such that in the case where the device may be used by multiple users, such as on an airplane or in a hotel, each user may have fresh sleeves. Although the term “sleeve” is used to describe the items numbered 44 and 46, these items may have any suitable construction, such as a strap-like construction.

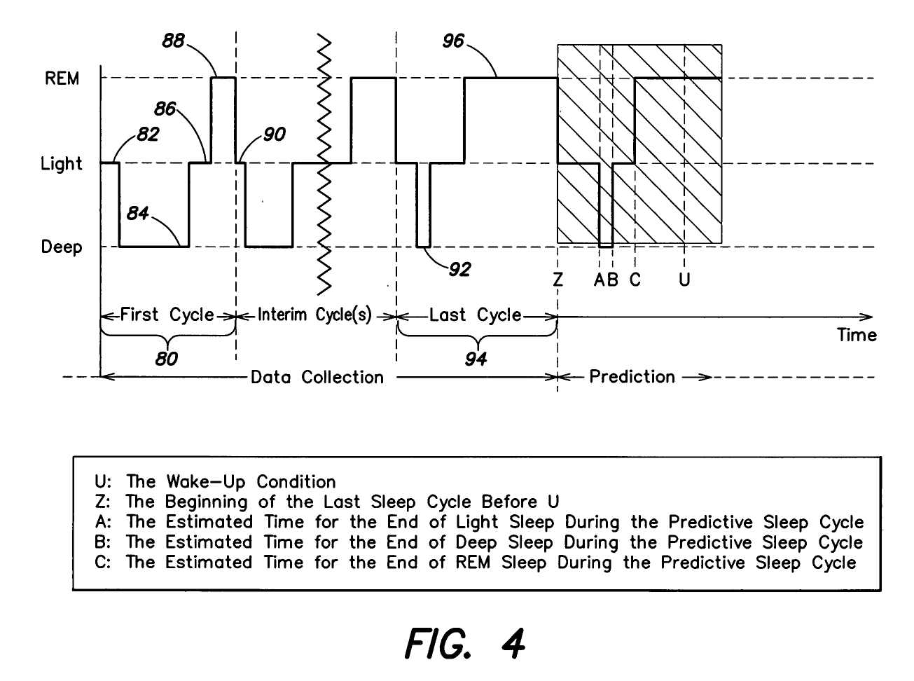

FIG. 4 shows a simplified hypnogram depicting a sleep pattern for an average user 10. When a user falls asleep, the user may enter a first sleep cycle 80 by going into light sleep 82. While in first sleep cycle 80, the user may progress into deep sleep 84, back to light sleep 86 and end the first sleep cycle 80 with a period in REM sleep 88. As the user shifts from REM sleep 88 to light sleep 90, the user may enter a next sleep cycle, and so on. This pattern of light-deep-light-REM sleep may repeat itself approximately every ninety minutes, although sleep cycle times may vary from person to person depending on a number of factors, such as age. Because this pattern repeats itself, it is possible to enter each sleep state a number of times throughout sleep. However, as a user sleeps, the amount of time spent in deep sleep may decrease, as can be seen in comparing deep sleep 84 of first sleep cycle 80 with deep sleep 92 of last sleep cycle 94, and the amount of time spent in REM sleep may increase, as can be seen in comparing REM sleep 88 of first sleep cycle 80 with REM sleep 96 of last sleep cycle 94. It is the repetition of sleep cycles and pattern of sleep states that may enable the monitoring portion 11 to predict when a user may enter a desired sleep state.

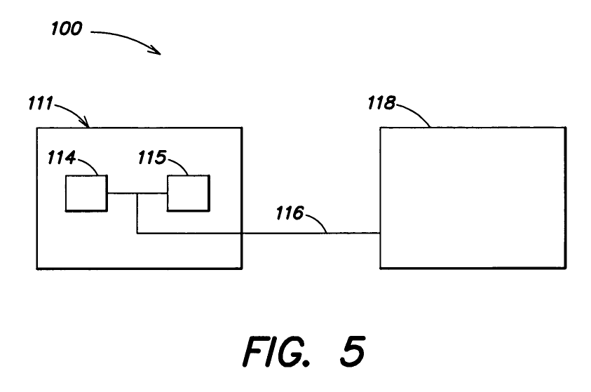

FIG. 5 shows a schematic block diagram of an embodiment of a wake-up device 100, including a monitoring portion 111 and an alarm portion 118. Monitoring portion 111 may include a sensing portion 114, having one or more sensors for monitoring a biosignal of a user, and a predicting portion 115.

Similar to monitoring portion 11, monitoring portion 111 may monitor at least one biosignal of a user. Although monitoring portion 11 is described as including a headband 12 in the embodiments in FIGS. 1-3, the sensing portion 114 is not so limited and may include a headpiece that encircles a portion of or the entire head of a user, a complete hat, discrete sensors that are removably adhered or otherwise secured to the head and/or other portions of a user, a neck piece, such as a device similar to a necklace or choker, a neck pillow, such as those used by airplane travelers, an eyeshade, a permanent implant underneath or proximate the skin of a user, or any other device or configuration that will allow the monitoring portion to monitor a biosignal of the user. In addition, the monitoring portion need not be placed contacting the head of a user and may be placed contacting or proximate another portion of the user, such as the neck, arms, legs, chest or any other portion of the user, as the present invention is not intended to be limited in these respects.

As described briefly above, in one embodiment, the sensing portion 114 may include electrodes. Electrodes may be placed against the skin of a user to monitor an electroencephalogram (EEG) signal, an electro-oculogram (EOG) signal and/or an electromyogram (EMG) signal. In addition or alternatively, temperature sensors, such as thermocouples or thermistors, or galvanic skin response meters may be placed adjacent the user to measure body temperature or resistance, respectively. It should be appreciated that other parameters and other placements of a sensing apparatus are possible, including measuring heart rates, breathing frequency, eye movement or other biophysiological properties, as the present invention is not intended to be limited in this respect.

Sensors may be composed of metal-coated or metal-infused fabric, such as silver-coated fabric, to contact the skin of the user. In addition or alternatively, the sensors may include carbon rubber, gold, or any other material or fabric.

The predicting portion 115 may assist in determining the sleep state of the user and may use current sensed information from the sensing portion 114, a sleep history of a user from prior sleep periods, a pre-determined hypnogram, similar to that described above in FIG. 4, sleep information of the user from earlier during sleep, such as a portion of a sleep cycle, a complete sleep cycle, or a plurality of sleep cycles, sleep information taken from another source, such as a tests, literature or other subjects, any combination thereof or any other device or information as long as predicting portion 115 may determine in what state of sleep the user currently is and/or what state of sleep the user may be in during a future time.

Thus, the predicting portion 115 may use any suitable data and/or algorithms, such as the wake-up algorithm described in the embodiment in FIG. 8 to predict when the user may be in the desired sleep state, and/or the sleep state determination algorithm described in FIG. 9 to determine the current sleep state of the user. As described above, inputs into the algorithm(s) may include raw or processed data from a sleep history of a user, earlier during sleep, sleep information taken from another source, or any other data. It should also be appreciated that the sleep state of the user may be actively monitored and this information may be used in actively readjusting any predicted occurrences, as the present invention is not intended to be limited in these respects.

In one embodiment, such as where a sleep history of a user or a pre-determined hypnogram is used to predict when the user may be in the desired sleep state, the sensing portion need not be included in the monitoring portion 111. For example, the functions of the sensing portion may be eliminated and the monitoring portion may be included in the same structure as the alarm portion.

In addition to sleep state information from sensing portion 114, a wake-up condition may be indicated to the predicting portion 115. Since the user will likely enter the desired sleep state more than once throughout the night, to guide the wake-up device in predicting during which occurrence of the desired sleep state the user wishes to be awakened, a wake-up condition may be determined. In one embodiment, the user may set a wake-up time at which or near to which the user wishes to be awakened. In another embodiment, the wake-up condition may include an interval during which the user wishes to be awakened, such as a time period before a defined wake-up time. In yet another embodiment, the wake-up condition may include both an interval and a wake-up time; the wake-up time representing a portion of the interval, such as the beginning, middle or end of the interval. It should be appreciated that the wake-up condition may be pre-programmed into the device or may be determined by the user or by a third party or any combination thereof, as the present invention is not intended to be limited in these respects.

Regardless of how the wake-up condition is defined, predicting portion 115 may predict at least one occurrence when a user will be in a desired sleep state that is at, near to, or during the wake-up condition. The predicted occurrence may be a moment in time or may be a window of time having a beginning, an end and a period therebetween. In one embodiment, a user may be awakened at the moment in time or at the beginning of the window. In another embodiment, the user may not be awakened until a portion of the window has elapsed, such as a percentage of the window (e.g., 25%, 50% or 63%) or a certain amount of time (e.g., 2, 5 or 7 minutes). In yet a further embodiment, the user may be awakened at the end of the window, as the present invention is not intended to be limited in these respects.

In one embodiment where the wake-up condition is an interval, the predicted occurrence may occur during the interval and the user may be awakened during that predicted occurrence regardless of whether the user is actually in the desired sleep state. In an alternative embodiment, monitoring portion 111 may determine the user's sleep state during the predicted occurrence to ensure that the user is in the desired sleep state. If the user is in the desired sleep state, the user may be awakened. If the user is not in the desired sleep state, the sensing portion 114 may continue to monitor the user's sleep state during a remainder period of the interval; the user may be awakened if the user enters the desired sleep state, or at the end of the interval, whichever occurs first.

In an alternative embodiment, the prediction of an occurrence when the user will be in the desired sleep state may be readjusted during sleep and/or during the interval. In these cases, either the user may be awakened during the readjusted prediction or the user's actual sleep state during the readjusted predicted occurrence may be determined to ensure that the user is now in the desired sleep state. It should be appreciated that predicting portion 115 may predict an occurrence of the desired sleep state once, when it is determined that the original prediction was inaccurate, and/or may be re-predicted or adjusted an infinite number of times, as the present invention is not intended to be limited in this respect.

The monitoring portion 111 may communicate with alarm portion 118 via communication link 116, for example, to indicate to the alarm portion 118 when to awaken the user. In one embodiment, monitoring portion 111 may only send an output to the alarm portion 118 once during each sleep; for example, where the output signals the alarm portion 118 to awaken the user. Alternatively, monitoring portion 111 may be in one or two-way communication with alarm portion 118 at any time, thereby working together to determine when to awaken the user.

In one embodiment, a user may input a wake-up condition into the alarm portion, which may then be communicated to the predicting portion. In addition or alternatively, the alarm portion may continually communicate information to the monitoring portion. In one embodiment, the sensing portion may only monitor the sleep state of the user at predetermined times throughout sleep. In this case, the alarm portion may send a signal to the monitoring portion indicating that it is time to monitor the user's sleep state. In addition, if a user is using the wake-up device while traveling to a different time zone, the user may only need to adjust the time on the alarm portion, and the alarm portion may then communicate the predetermined monitoring times to the sensing portion with the time zone adjustment already taken into account.

It should be appreciated that the alarm portion may communicate with the monitoring portion via wires, such as a dedicated line, wireless transmission, such as infrared line of sight, Bluetooth, cellular, microwave, satellite, and radio waves, or any other means. In an alternative embodiment, the alarm and monitoring portions may be integrated into one unit, such that there may be no need for exterior transmission of data. The interaction between the monitoring portion and the alarm portion may be one or two way communication and may occur once, twice, during an interval, periodically, or constantly, as the present invention is not intended to be limited in these respects.

After receiving an output from monitoring portion 111, alarm portion 118 may awaken the user. Although as shown in the embodiments described in FIGS. 1-3, the alarm portion is located proximate, but not physically contacting the monitoring portion, it should be appreciated that the alarm portion may be located on or adjacent the monitoring portion, such as in bulges 50 on headband 12, and/or proximate or contacting the user, as the present invention is not limited in this respect.

Alarm portion 118 may awaken the user using a variety of methods. In one embodiment, the user may be awakened by a conventional alarm, including an auditory alert, such as a radio or buzzer, or a visual alert, such as a flashing, strobe or bright light. In addition or alternatively, the user may be awakened by a smell, a change in temperature, vibrations, a change in hemodynamics, another environmental control or any combination thereof, as the present invention is not intended to be limited in this manner.

In addition or alternatively, the user may be awakened using sleep modification techniques, wherein an environmental factor may be adjusted to gradually modify the sleep state of the user to awaken the user. In one embodiment, circadian rhythm modification may be accomplished by using a light that is colored to a wavelength optimal for modifying a user's circadian rhythms. Sleep modification techniques may also be used to prevent a user from entering a non-desired sleep state. For example, sound may be used to prevent a user from entering deep sleep before awakening the user. A variety of inducing factors may be used, such as sound, vibration, hemodynamics (e.g., by changing a user's body angle, thus the user's blood pressure), temperature (e.g., heating or cooling a user's neck, forehead or any other body part), light, electrical or magnetic stimulation, or any combination thereof, as the present invention is not intended to be limited in these respects.

Alarm portion 118 may also perform additional functions and contain additional accoutrements. A speaker may be used to project sound to awaken the user from sleep. An AM/FM radio, cassette, CD or MP3 player and the appropriate controls may be included with alarm portion 118 for projection via the speaker. Alarm portion 118 may also contain a light which may be activated at desired times, as determined by monitoring portion 111, to expose the user to light. Light exposure may inhibit melatonin production; therefore, a user may be more likely to awaken gracefully after being exposed to light.

Alarm portion 118 may have a display, such as an LED display, for displaying standard clock information, such as alarm time or actual time. The display may also be used to show sleep-related information. For example, a colored light may indicate the current phase of sleep of a user so that a third party, such as another person or a computer, may decide when to wake up a user based on the user's sleep state. The display may also be used to show the total time spent sleeping and/or the total time spent in each phase of sleep that the user experience during the previous night. The sleep information may also be displayed in the form of a hypnogram.

Alarm portion 118 may have a number of user input buttons along with the standard clock inputs. Button inputs for wake-up time, optimal or short wake-up intervals, nap mode, light socket mode (on, off or automatic as decided by the processor), as well as configurations of different settings such as snooze time (in minutes), wake-up interval (in minutes), and/or minimum or maximum number of sleep cycles may all be included as inputs. In embodiments wherein the alarm portion may be integrated into the monitoring portion, some or all functions of the alarm portion may be performed by the monitoring portion.

Alarm portion 118 may also include a storage space or a holder for monitoring portion 111 when monitoring portion 111 or the entire wake-up device is not in use. In one embodiment wherein monitoring portion 111 includes a rechargeable battery, alarm portion 118 may also contain a charging station for monitoring portion 111. In addition, alarm portion 118 may include an LED for indicating the charging status of monitoring portion 111. In an alternative embodiment, alarm portion 118 and/or monitoring portion 111 may be powered and/or recharged using a wall plug.

An aspect of the wake-up device will now be described with reference to a headband unit and an alarm clock unit. It should be appreciated that aspects of the invention are not intended to be limited to use with this specific embodiment, but rather that the monitoring and alarm portions of the wake-up device may be implemented in any suitable way.

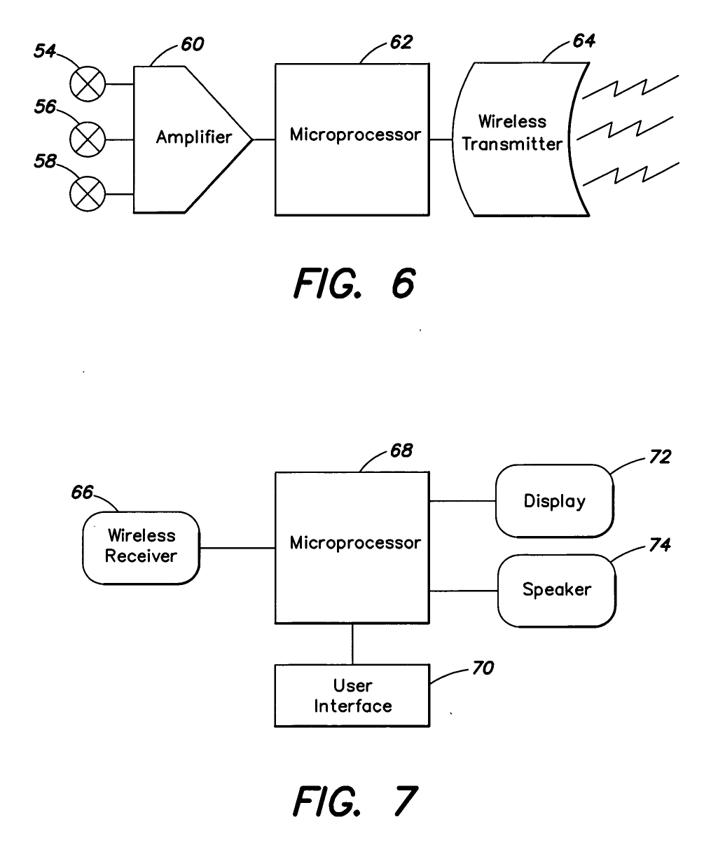

As shown in the embodiment depicted in FIG. 6, the headband unit may include three electrodes 54, 56, 58. Signals from these electrodes 54, 56, 58 may connect to a circuit board (not shown) and may be amplified by amplifier 60. Amplifier 60 may use a large gain to bring the differential between signal electrodes 54, 56 up to a level where it can be used as an input into an Analog to Digital Converter (ADC), which, in some embodiments, may be integrated into a microprocessor 62. The amplified signals may be converted to a digital signal by the ADC, which may use a Right Leg Driver (DRL) system to eliminate common mode noise.

The digital signal may be read by microprocessor 62 at defined signal transit times. The microprocessor 62 may determine the transmit times based on predetermined values and may wirelessly transmit the digital signal to the alarm clock unit, an embodiment of which is shown in FIG. 7, using the wireless transmitter 64. Microprocessor 62 may arrange wireless transmission times to minimize power usage. In addition, the wireless transmitter may be, but need not be, integrated onto microprocessor 62. Alternatively, in some embodiments, wireless transmission may not be necessary.

As shown in the embodiment shown in FIG. 7, the alarm clock unit may include a wireless receiver 66 to communicate with the headband unit, for example, using electromagnetic waves for transmission. A Digital Signal Processor (DSP) 68 on the alarm clock may analyze the received data from the headband according to the sleep state detection algorithm detailed in FIG. 9. The DSP 68 may also run the wake-up algorithm, and decide when to awaken the user. (Thus, in this embodiment, the alarm clock unit may include the predicting portion 115 of the monitoring portion 111 and the alarm portion 118, whereas the headband unit includes the sensing portion 114.)

The microprocessor 68 may contain within its memory both a method for determining sleep state, for example, as detailed in FIG. 9, and a method for determining wake-up time, for example, as detailed in FIG. 8. Also contained within the memory of microprocessor 68 may be parameters set by the user through a user interface 70. For possible use in the methods of determining sleep state and wake-up, these parameters may include the wake-up condition, such as the wake-up time and/or the interval, and possibly other useful inputs. Microprocessor 68 may drive an output display 72 similar to that of a standard alarm clock, and may control a speaker 74 to awaken the user at the appropriate time. The method for awakening the user with a speaker may be replaced with a method using vibration, light or any other means to awaken the user, as described above.

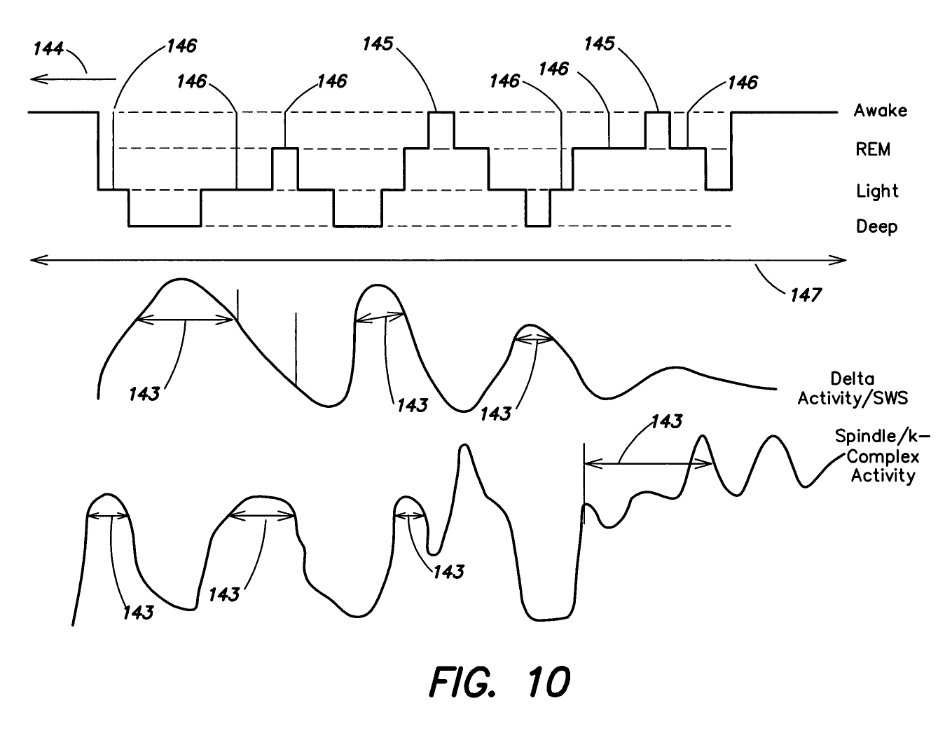

As shown in the embodiment depicted in FIGS. 8 and 10, a predictive wake-up algorithm may accept a user-defined wake-up condition in step S122 as an input. During sleep states S123, sleep data in step S124 may be stored in the memory of a microcontroller or a microprocessor. Sleep data and a determination that a user is at the end of a sleep cycle in step S125 may assist in deriving a first subset of parameters including, but not limited to, sleep efficiency or sleep efficiency index (e.g., total time spent sleeping divided by the total time spent in bed), mean sleep latency (e.g., averaged sleep onsets), sleep cycle length, sleep state latencies 143, percentage of a sleep state during sleep, percentage of a sleep state during a sleep cycle, sleep onset 144, intra sleep wakefulness (e.g., the total time spent moving and awake divided by the total time spent in bed), total movement time, number of awakenings 145, number of movement arousals 146, total sleep time, or total time in bed 147, as shown in the embodiment in FIG. 10.

Entrance into the last sleep cycle in step S126 may be determined using a variety of factors, such as wake-up time, sleep data from a portion of or an entire sleep cycle or from a plurality of sleep cycles, and/or sleep history. The above-mentioned parameters may be stored, for example, in a memory of a microprocessor, and may be compared to comparable parameters determined using a different algorithm, for example, the sleep state detection algorithm in the embodiment of FIG. 9.

In step S127, additional parameters derived from the trends of sleep cycles may help estimate the last sleep cycle, including but not limited to the end of light sleep, the end of deep sleep, and the end of REM sleep. Using parameters from step S127, whether or not the end of light is after the wake-up condition may be determined in step S129. If it is determined that the end of light sleep is after the wake-up condition in step S130, the wake-up condition may be awaited in step S131 before awakening the user in step S132. If it is determined that the end of light sleep is not after the wake-up condition in step S133, it will be determined whether or not the end of deep sleep is after the wake-up condition in step S134. If it is determined that the end of deep sleep is after the wake-up condition in step S135, then the end of light sleep will be awaited in step S136 before awakening the user in step S132. If it is determined that the end of deep sleep is not after the wake-up condition in step S137, it will be determined whether or not the end of REM sleep is after the wake-up condition in step S138. If it is determined that the end of REM sleep is after the wake-up condition in step S139, then the end of deep sleep will be awaited in step S140 before awakening the user in step S132. If it is determined that the end of REM sleep is not after the wake-up condition in step S141, the wake-up condition will be awaited in step S142 before awakening the user in step S132.

All of the above-mentioned parameters may be determined by statistical and signal processing techniques including, but not limited to, linear predication, Kalman filtering, time series estimations, Markov chains, regressions, non-parametric statistics and neural networks. These techniques may be based on, but not limited to, characteristics of light, deep and/or REM sleep.

An embodiment of a sleep state detection algorithm used by the wake-up device is shown in the embodiment depicted in FIG. 9. One channel of differential input from the two signal electrodes may be taken as a signal input 22. Feature vector construction 24 may select features of the signal, such as power in the delta band (0.5-4 Hz), power in the theta band (5-8 Hz), power in the sigma band (11-14 Hz), power in the alpha band (8.5-12 Hz), power in the gamma band (12-30 Hz), power in an arbitrary frequency band within the 1-30 Hz band, the ratio of any 2 frequency bands, sleep spindle amplitude, sleep spindle density, K-Complex detection, chaos parameters, or any other feature of the signal. The output 25 of the feature vector construction 24 may be inputted into an artificial neural network 26. The neural network 26 may have previously been trained, for example, by professionally scored sleep data, to perform sleep scoring on the signal at regular intervals, such as on a 30 second epoch. The output of the neural network may be a set of weights 27 indicating sleep state, such as sleep stages 1, 2, 3, 4, REM, move and wake. A post processing stage 28 may apply appropriate rules to the current epoch using previous and future epoch determination to further improve the final sleep state determination 30, which may be used in the wake-up algorithm in FIG. 8.

In addition to the method described above, a variety of sleep state detection means may be used, such as hidden mark-up models or processing systems similar to those set forth in A Manual of Standardized Terminology: Techniques and Scoring Stages of Human Subjects, by Rechtschaffen and Kales, as the present invention is not intended to be limited in this respect.

It should be appreciated that sleep modification techniques may also be exercised while a user is falling asleep or sleeping and are not limited to awakening a user. In some embodiments, sleep modification techniques may be used to modify sleep onset, micro and macro sleep architecture and wake-up modification of the present night or future nights. For example, if it is predicted that a user will be entering the desired sleep state right after the wake-up interval ends, a sleep cycle of the user may be induced, thereby shifting the user's sleep state so that the user may enter the desired sleep state during the wake-up interval, rather than after the wake-up interval ends. In an alternative embodiment, a user's hands and feet may be heated while the user's core may be cooled to help induce sleep, thereby assisting a user who may have difficulties falling asleep.

In addition or alternatively, future nights' sleep cycles may be altered, for example, by shifting circadian rhythms. This effect may be achieved by incorporating a light source, such as an LED, capable of emitting light, such as a super blue colored light (462 nm wavelength), which would be turned on at the appropriate times during sleep as determined by an algorithm, for example, by the phase response curve detailed in U.S. Pat. No. 5,545,192, which is hereby incorporated by reference in its entirety, or by another method. Shifting the circadian rhythms of a user may cause the user to fall asleep earlier or later the next night. In one embodiment, this circadian rhythm manipulation may be used to combat jet lag.

It should be appreciated that any sleep state information that has been collected through monitoring a user's sleep states or any other means may be used in determining which sleep modification technique or techniques to use and/or the timing and intensity thereof.

Although frequently described above as being light sleep, the desired sleep state may be any state of sleep in which a user wishes to be awakened. To minimize sleep inertia, awakening during light sleep (i.e., sleep stages 1 and 2) may be desired. However, awakening during other sleep states, such as deep sleep (i.e., sleep stages 3 and 4) or REM (Rapid Eye Movement) sleep, may also be desired. For example, if a user wishes to remember the user's dreams more vividly and/or accurately, awakening during REM sleep may be desirable, as the present invention is not intended to be limited in this respect.

In one embodiment, the user may choose in which sleep state the user should be awakened. For example, there may be a switch that a user may move between options signifying light sleep, deep sleep, and REM sleep to indicate in which sleep state the user would like to be awakened. In an alternative embodiment, the wake-up device may be pre-programmed to wake the user in a certain sleep state, such as light sleep, as the present invention is not limited in this respect.

As described briefly above, the wake-up condition may be any condition which indicates to the wake-up device during which occurrence of the desired sleep state the user wishes to be awakened. Similar to a typical alarm clock, the user may set a wake-up time at which or near to which the user wishes to be awakened. As described above, the wake-up time may represent the latest time until which the user wishes to sleep. Although the user may be awakened before this wake-up time, in one embodiment, the user may be awakened at the wake-up time, even if the user is not in the desired sleep state at the wake-up time. In an alternate embodiment, the wake-up time may represent the first time after which the user wishes to be awakened. Although the user may be awakened after this wake-up time, the wake-up device should not awaken the user before this wake-up time. It should be appreciated that the user may be awakened at, before or after the wake-up time as the present invention is not intended to be restricted in this manner.

While the wake-up time may be indicated by an exact time, such as 7:15am, 6:43am or 10:30am, the wake-up time may also be indicated by any measure, such as maximum sleeping time or minimum sleeping time (e.g., 6 hours, 7.5 hours or 8 hours). Maximum and minimum sleeping times may be calculated from the time that the user actually spent asleep, from the time that the user spends in bed or lying down, or from sleep quality measures.

In one embodiment, the user may choose a napping mode, wherein the user may be awakened at the end of an optimal nap time, such as 20 minutes. The optimal nap time may be set so that the user may avoid entering deep sleep; however, other times may be used. In addition, the user may set a wake-up time, so that the user may not have to worry about not being awakened by an appropriate time. For example, if a user has a 4pm appointment and lies down for a nap at 3pm, but does not actually fall asleep until 3:30pm; the user may set a wake-up time of 3:55pm, so that the user will not oversleep.

In another embodiment, the wake-up condition may be an interval during which the user wishes to be awakened. The interval may be a time period, such as from 6:30am to 7:00am, wherein the user may be awakened during this interval when the user is in the desired sleep state. In one embodiment, the user may enter the desired sleep state more than once during this interval; in this case, the user may be awakened during the last occurrence of the desired sleep state, thereby allowing the user to sleep as long as possible, while still awakening the user in the desired sleep state. In an alternative embodiment, the user may be awakened during the first, second or any other occurrence of the desired sleep state, as the present invention is not limited in this respect. For example, a user may wish to be awakened during the fifth time that the user is in light sleep.

It should be appreciated that the interval may be pre-programmed into the wake-up device or may be determined by the user, as the present invention is not intended to be limited in this respect. Similar to the wake-up time, the interval may be represented by exact start and finish times, such as 6:00am-6:45am or 8:17am-8:32am, or may be represented by an interval of time, such as 15, 30, 37 or 45 minutes, as the present invention is not limited in this manner.

In a related embodiment, the wake-up condition may be represented by both a wake-up time and an interval, in that the wake-up time may represent the beginning, middle or end of the interval. In one embodiment, the wake-up device may be pre-programmed to wake the user during a 30 minute interval and the user may set a wake-up time, representing the end of the interval. For example, if the user chooses a wake-up time of 6:45am, the user will be awakened between 6:15am and 6:45am. In another embodiment, the user may choose between two or more pre-programmed intervals, such as 15, 30, or 45 minutes, and may set the wake-up time, thereby allowing the user more options.

The wake-up condition need not relate to time, but may be dependent upon sleep cycles. These options may allow a user to discover the user's own sleeping patterns and needs and adjust the user's wake-up device accordingly. For example, the user may choose to be awakened during the fifth sleep cycle, such that the wake-up device may predict when during the fifth sleep cycle, the user will be in the desired sleep state and awaken the user during that predicted occurrence. In an alternative embodiment, the user may find that to be able to function satisfactorily during the day, the user requires three cycles of deep sleep; therefore, the user may choose to be awakened after the user has experienced three cycles of deep sleep. In yet another embodiment, a user may find that the user's fourth dream of the night is usually a nightmare and may choose to be awakened before the user's fourth REM cycle.

It should be appreciated that the sensed and/or predicted information may be transferable to another device for analysis or storage. For example, the information may be transmitted to a computer, such as the user's personal computer. The user may store and/or study the data on the user's own. In addition or alternatively, the information may be sent to a sleep lab or other processing facility for analysis and/or storage.

It should be further appreciated that although some embodiments of the invention are directed to “determining an actual sleep state” of a user, any measurement or parameter that correlates to a user's sleep state, without necessarily having to determine whether the user is in light, deep or REM sleep, is contemplated. For example, a wake-up device may determine a user's temperature and by inputting the user's temperature into an algorithm, predict when the user should be woken without actually determining the sleep state (e.g., light, deep, or REM) of the user. Further, the “desired sleep state” may also be a measurement or parameter that correlates to a user's sleep state. For example, the desired sleep state may be a temperature or a temperature range, such that the user may desire to be awakened when the user's body temperature is in a certain range or near a certain temperature. It should be appreciated that the parameter or measurement is not limited to temperature and may include resistance, heart rate, breathing frequency, other parameters as described above and any parameter or measurement that correlates to sleep.

In one embodiment, a sleep monitoring and alarm system may include: a detection unit with contact points on the subject to detect different biophysiological signals which are indicative of one's sleep state; an electronics circuit board contained within the detection unit, including a number of electrodes or other methods for signal detection, amplification circuitry to condition the signals, and a method for transmission, such as wireless transmission, of the signal data; a power source contained within the detection unit for powering the electrical components of the unit; an alarm clock unit in communication with the detection unit, although the alarm clock unit may be integrated into the detection unit; an electronics circuit board contained within the alarm clock unit including a wireless receiver for communications with the detection unit, and a digital signal processor for implementation of certain algorithms; and a set of components for standard alarm clock features including a display, buttons for user interaction, and a speaker, light, or other method of awakening the user in communication with the processor. The detection unit may collect biophysiological signals indicative of sleep state, condition those signals, and transmit them to the alarm clock unit. The alarm clock unit may process these signals using a set of sleep state algorithms to determine the current sleep state and to analyze sleep data using a predictive algorithm to determine the wake-up point. The alarm clock may awaken the user based on a wake-up time algorithm during a phase of sleep optimal for the user's performance. This embodiment may not preclude integration of the alarm clock unit into the same physical housing of the detection unit for ease of use.

In addition or alternatively, the clock unit may have a display mode which may show how much time the user spent in REM, light, or deep sleep during the night.

In addition or alternatively, the alarm unit may not be used to modify the sleep behavior of the user, but to provide biophysiological information for medical or non-medical use.

In addition or alternatively, the detection unit may include a method of waking-up the user, but not disturbing others in the environment.

In addition or alternatively, multiple detection units may be connected to multiple users and the system may monitor and modify biophysiological patterns in all users independently.

In addition or alternatively, the alarm unit may emit a chirp of sound during the night to determine sleep stage. In response to the chirp of sound, the EOG signal on the user may show a spike due to eye movement triggered by the noise, indicating that the user may be in stage 1 and/or stage 2 sleep. In these stages of sleep, the user may be partially aware and able to react to the surroundings. Other signals, such as an EMG signal, may be used to determine sleep stage from this stimulus. Any results may be used in the wake-up algorithm.

In another embodiment, a method for reducing or eliminating sleep inertia in a subject may include awaking the subject from sleep using any of the devices and/or programs described above.

In another embodiment, a method for awaking a subject from sleep may include detecting and analyzing biophysiological impulses from a sleeping subject using any of the devices and/or programs described above.

Various aspects of the invention include the following:

A1. A method of predicting a sleep state of a user comprising:

monitoring at least a period of a sleep cycle of a user;

predicting a time period during which the user will be in a desired sleep state and/or a sleep state the user will be in during a desired time period.

A2. The method of claim A1, wherein the monitoring step includes determining a sleep state of a user during the at least the period of the sleep cycle.

B1. A method of waking a user comprising:

determining a wake-up condition relating to when a user will be awakened;

predicting at least one occurrence when the user will be in a desired sleep state, the predicted at least one occurrence being approximately at, near to, or during the wake-up condition; and

waking the user.

B2. The method of claim B1, wherein the desired sleep state includes light sleep.

B3. The method of claim B2, wherein light sleep includes at least one of sleep stages 1 or 2.

B4. The method of claim B1, wherein the desired sleep state includes REM sleep.

B5. The method of claim B1, wherein the predicted at least one occurrence includes a period of time.

B6. The method of claim B1, wherein the wake-up condition includes a point in time.

B7. The method of claim B6, wherein the predicted at least one occurrence is after the point in time.

B8. The method of claim B1, wherein the wake-up condition includes an interval, the interval having a beginning and an end.

B9. The method of claim B8, wherein the interval includes a time period.

B10. The method of claim B9, wherein the time period is less than approximately an hour.

B11. The method of claim B10, wherein the time period is less than approximately fifteen minutes.

B12. The method of claim B8, wherein the interval includes a sleep cycle.

B13. The method of claim B8, wherein waking the user includes waking the user during the interval.

B14. The method of claim B13, wherein waking the user during the interval includes waking the user at the end of the interval.

B15. The method of claim B1, wherein determining the wake-up condition includes allowing the user to choose a point in time and at least one of at least two predetermined intervals.

B16. The method of claim B15, wherein the at least two predetermined intervals include a first predetermined interval of approximately 40 minutes and a second predetermined interval of approximately 15 minutes.

B17. The method of claim B1, wherein waking the user includes waking the user during the predicted at least one occurrence.

B18. The method of claim B17, wherein waking the user during the predicted at least one occurrence includes waking the user after approximately more than 50% of the predicted at least one occurrence has elapsed.

B19. The method of claim B18, wherein the predicted at least one occurrence has a beginning and an end, and wherein waking the user after approximately more than 50% of the predicted at least one occurrence has elapsed includes waking the user at approximately the end of the predicted at least one occurrence.

B20. The method of claim B1, wherein predicting the at least one occurrence includes:

determining a sleep state of a user before the beginning of the wake-up condition; and

using sleep state information from the determining step in predicting the at least one occurrence.

B21. The method of claim B1, wherein predicting the at least one occurrence includes using a sleep history of a user in predicting the at least one occurrence.

B22. The method of claim B1, further comprising:

determining a sleep state of the user during the predicted at least one occurrence; and

determining if the sleep state during the predicted at least one occurrence is the desired sleep state.

B23. The method of claim B22, wherein, if it is determined that at any point the sleep state during the predicted at least one occurrence is the desired sleep state, waking the user includes waking the user after making the determination that the sleep state during the predicted at least one occurrence is the desired sleep state.

B24. The method of claim B22, wherein, if it is determined that the sleep state during the predicted at least one occurrence is not the desired sleep state, waking the user includes:

determining a sleep state of the user at least after the predicted at least one occurrence;

determining if the sleep state after the predicted at least one occurrence is the desired sleep state; and

waking the user after the predicted at least one occurrence.

B25. The method of claim B24, wherein, if it is determined that at any point the sleep state after the predicted at least one occurrence is the desired sleep state, waking the user includes waking the user after making the determination that the sleep state after the predicted at least one occurrence is the desired sleep state.

B26. The method of claim B22, wherein the wake-up condition includes an interval, the interval having a beginning and an end, and wherein, if it is determined that the sleep state during the predicted at least one occurrence is not the desired sleep state, waking the user includes:

determining a sleep state of the user at least during a remainder period, the remainder period being after the predicted at least one occurrence and before the end of the interval;

determining if the sleep state during the remainder period is the desired sleep state; and

waking the user during the remainder period.

B27. The method of claim B26, wherein, if it is determined that the sleep state during the remainder period is not the desired sleep state, waking the user includes waking the user at the end of the interval.

B28. The method of claim B22, wherein the wake-up condition includes an interval, the interval having a beginning and an end, and wherein, if it is determined that the sleep state during the predicted at least one occurrence is not the desired sleep state, waking the user further includes waking the user at the end of the interval.

B29. A wake-up device for performing the method of claim B1.

B30. The wake-up device of claim B29, wherein the wake-up device comprises:

a monitoring portion producing an output; and

an alarm portion to assist in awakening the user based on the output.

B31. The wake-up device of claim B30, wherein the monitoring portion is adapted to be worn on a head of the user and the alarm portion is adapted to be placed proximate, but not physically contacting the user.

B32. The wake-up device of claim B30, wherein the monitoring portion includes at least one sensor for monitoring at least one biosignal of the user.

B33. The wake-up device of claim B32, wherein the at least one sensor is an electrode.

B34. The wake-up device of claim B30, wherein the monitoring portion performs at least the predicting step and the alarm portion performs at least the waking step.

B35. The wake-up device of claim B30, wherein the method performed by the wake-up device further includes:

determining a sleep state of the user during the predicted at least one occurrence; and determining if the sleep state during the predicted at least one occurrence is the desired sleep state;

and wherein the monitoring portion performs at least the determining a sleep state of the user step and the alarm portion performs at least the waking step.

B36. The wake-up device of claim B35, wherein the monitoring portion further performs the predicting step and the determining if the sleep state during the predicted at least one occurrence is the desired sleep state step.

C1. A wake-up device comprising:

a monitoring portion to monitor at least one biosignal of a user during at least a portion of a sleep cycle of a user and to predict a time period during which the user will be in a desired sleep state and/or a sleep state in which the user will be during a desired time period, the monitoring portion producing an output; and

an alarm portion to receive the output and to assist in awakening the user in response to the output.

C2. The wake-up device of claim C1, wherein the monitoring portion includes a wake-up condition and the predicted time period and/or predicted sleep state is near to or during the wake-up condition.

C3. The wake-up device of claim C2, wherein the wake-up condition is user-definable.

C4. The wake-up device of claim C2, wherein the wake-up condition includes an interval and the predicted time period and/or predicted sleep state is during the interval.

C5. The wake-up device of claim C2, wherein the wake-up condition includes a time and the predicted time period and/or predicted sleep state is near to the time.

C6. The wake-up device of claim C1, wherein the monitoring portion determines a sleep state of the user during the predicted time period and/or predicted sleep state and determines if the sleep state during the predicted time period and/or predicted sleep state is the desired sleep state.

C7. The wake-up device of claim C1, wherein, if it is determined that at any point the sleep state during the predicted time period and/or predicted sleep state is the desired sleep state, the monitoring portion produces a wake-up output.

C8. The wake-up device of claim C1, wherein the monitoring portion includes at least two sensors for monitoring the at least one biosignal of the user.

C9. The wake-up device of claim C1, wherein the monitoring portion includes a headpiece, the at least one sensor being located on the headpiece.

C10. The wake-up device of claim C9, wherein the alarm portion is located on the headpiece.

C11. The wake-up device of claim C9, wherein, when in use, the headpiece is constructed and arranged to locate the sensors on a forehead of the user.

C12. The wake-up device of claim C1, wherein the monitoring portion communicates wirelessly with the alarm portion.

C13. The wake-up device of claim C1, wherein the alarm portion includes an alarm.

C14. The wake-up device of claim C13, wherein the alarm includes a sound.

C15. The wake-up device of claim C13, wherein the alarm includes a light.

C16. The wake-up device of claim C1, wherein the alarm portion is adapted to be proximate, but not physically contacting the user.

C17. The wake-up device of claim C1, wherein the monitoring device is adapted to be proximate the alarm portion.

C18. The wake-up device of claim C17, wherein the monitoring device is adapted to be physically contacting the alarm portion.

C19. The wake-up device of claim C1, wherein the at least one biosignal includes an electroencephalogram (EEG) signal, an electro-oculogram (EOG) signal or an electromyogram (EMG) signal or any combination thereof.

D1. A method of placing sensors to measure at least one biosignal of a user comprising:

placing a first sensor at a first location on a head of a user; and

placing a second sensor at a second location on the head of the user;

wherein the first location is approximately less than 10, 9, 8, 7, 6, 5, 4, 3, 2, 1, or any fraction thereof inches or centimeters from the second location or is less than a percent distance from the second location.

E1. A use of a fabric comprising a metal, such as silver, in the manufacture of a material for a sensor for monitoring a sleep state of a user.

The foregoing written specification is to be considered to be sufficient to enable one skilled in the art to practice the invention. While the best mode for carrying out the invention has been described in detail, those skilled in the art to which this invention relates will recognize various alternative embodiments including those mentioned above as defined by the following claims. The examples disclosed herein are not to be construed as limiting of the invention as they are intended merely as illustrative of particular embodiments of the invention as enabled herein. Therefore, systems and methods that are functionally equivalent to those described herein are within the spirit and scope of the claims appended hereto. Indeed, various modifications of the invention in addition to those shown and described herein will become apparent to those skilled in the art from the foregoing description and fall within the scope of the appended claims.

What is claimed is:

1. A method of waking a user comprising:
determining a wake-up condition relating to when a user will be awakened;
predicting at least one occurrence when the user will be in a desired sleep state, the predicted at least one occurrence being approximately at, near to, or during the wake-up condition; and
waking the user.

2. The method of claim 1, wherein the wake-up condition includes an interval, the interval having a beginning and an end and wherein waking the user includes waking the user during the interval.

3. The method of claim 2, wherein determining the wake-up condition includes allowing the user to choose a point in time to define the end of the interval.

4. The method of claim 1, wherein predicting the at least one occurrence includes:
determining a sleep state of a user before the beginning of the interval; and
using sleep state information from the determining step in predicting the at least one occurrence.

5. The method of claim 1, further comprising:
determining an actual sleep state of the user during the predicted at least one occurrence; and
determining if the actual sleep state during the predicted at least one occurrence is the desired sleep state.

6. The method of claim 5, wherein, if it is determined that at any point the actual sleep state during the predicted at least one occurrence is the desired sleep state, waking the user includes waking the user after making the determination that the actual sleep state during the predicted at least one occurrence is the desired sleep state.

7. The method of claim 5, wherein, if it is determined that the actual sleep state during the predicted at least one occurrence is not the desired sleep state, waking the user includes:
determining an actual sleep state of the user at least after the predicted at least one occurrence;
determining if the actual sleep state after the predicted at least one occurrence is the desired sleep state; and
waking the user after the predicted at least one occurrence;
and wherein, if it is determined that at any point the actual sleep state after the predicted at least one occurrence is the desired sleep state, waking the user includes waking the user after making the determination that the actual sleep state after the predicted at least one occurrence is the desired sleep state.

8. The method of claim 5, wherein the wake-up condition includes an interval, the interval having a beginning and an end, wherein, if it is determined that the actual sleep state during the predicted at least one occurrence is not the desired sleep state, waking the user includes:
determining an actual sleep state of the user at least during a remainder period, the remainder period being after the predicted at least one occurrence and before the end of the interval;
determining if the actual sleep state during the remainder period is the desired sleep state; and
waking the user during the remainder period;
and wherein, if it is determined that the actual sleep state during the remainder period is not the desired sleep state, waking the user includes waking the user at the end of the interval.

9. The method of claim 5, wherein the wake-up condition includes an interval, the interval having a beginning and an end, and wherein, if it is determined that the actual sleep state during the predicted at least one occurrence is not the desired sleep state, waking the user further includes waking the user at the end of the interval.

10. A wake-up device for performing the method of claim 1.

11. The wake-up device of claim 10, wherein the wake-up device comprises:
a monitoring portion producing an output; and
an alarm portion to assist in awakening the user based on the output.

12. The wake-up device of claim 11, wherein the monitoring portion is adapted to be at least partially worn on a head of the user and the alarm portion is adapted to be placed proximate, but not physically contacting the user.

13. The wake-up device of claim 11, wherein the monitoring portion includes at least one sensor for monitoring at least one biosignal of the user.

14. The wake-up device of claim 11, wherein the monitoring portion performs at least the predicting step and the alarm portion performs at least the waking step.

15. The wake-up device of claim 11, wherein the method performed by the wake-up device further includes:
determining an actual sleep state of the user during the predicted at least one occurrence; and
determining if the actual sleep state during the predicted at least one occurrence is the desired sleep state;
and wherein the monitoring portion performs at least the determining an actual sleep state of the user step and the alarm portion performs at least the waking step.

16. A wake-up device comprising:
a monitoring portion to monitor at least one biosignal of a user during at least a portion of a sleep cycle of a user and to predict a time period during which the user will be in a desired sleep state, the monitoring portion producing an output; and
an alarm portion to receive the output and to assist in awakening the user in response to the output.

17. The wake-up device of claim 16, wherein the monitoring portion includes a wake-up condition and the predicted time period is near to or during the wake-up condition.

18. The wake-up device of claim 17, wherein the wake-up condition includes an interval, having a beginning and an end, and a point in time, wherein the point in time is user-definable and represents the end of the interval.

19. The wake-up device of claim 17, wherein the wake-up condition includes an interval and the predicted time period is during the interval.

20. The wake-up device of claim 16, wherein the monitoring portion determines an actual sleep state of the user during the predicted time period and determines if the actual sleep state during the predicted time period is the desired sleep state.

21. The wake-up device of claim 16, wherein, if it is determined that at any point the sleep state during the predicted time period is the desired sleep state, the monitoring portion produces a wake-up output.

22. The wake-up device of claim 16, wherein the monitoring portion includes a headpiece and at least one sensor located on the headpiece.

23. The wake-up device of claim 22, wherein at least a portion of the alarm portion is located on the headpiece.

24. The wake-up device of claim 22, wherein, when in use, the headpiece is constructed and arranged to locate the at least one sensor on a forehead of the user.

25. The wake-up device of claim 16, wherein the monitoring portion communicates wirelessly with the alarm portion.

26. The wake-up device of claim 16, wherein the alarm portion is adapted to modify a user's sleep state to assist in awakening the user.

27. The wake-up device of claim 26, wherein the alarm portion includes an alarm that changes at least one of sound, light, vibration, temperature, hemodynamics, electricity or magnetism or any combination thereof.

28. The wake-up device of claim 16, wherein the at least one biosignal includes an electroencephalogram (EEG) signal, an electro-oculogram (EOG) signal or an electromyogram (EMG) signal or any combination thereof.

29. Use of fabric comprising a metal in the manufacture of a material for a sensor for monitoring a sleep state of a user.

30. The use of claim 29, wherein the metal includes silver.
